SEQUENCE LISTING (3 sequences)

SEQ ID NO: 1 - protein, 1254 aa, synthetic construct
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 2 - protein, 580 aa, synthetic construct
KRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 3 - DNA, 1746 bp, synthetic construct
  source 1..1746: mol_type = other DNA
atgaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaagggccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtaa

(19) United States
(12) Patent Application Publication
(10) Pub. No.: US 2026/0263574 A1
(43) Pub. Date: Sep. 10, 2026

(54) COMBINATION THERAPY OF HER2 VACCINE AND IMMUNE CHECKPOINT INHIBITOR

(71) Applicant: Aston Science Inc., Seoul (KR)

(72) Inventors: Hun Jung, Seoul (KR); Hyo Hyun Park, Gyeonggi-do (KR); Jee Hyun Choi, Gyeonggi-do (KR); Jin Ho Kang, Gyeonggi-do (KR); Eun Kyo Joung, Seoul (KR)

(21) Appl. No.: 19/162,333

(22) PCT Filed: Feb. 27, 2024
(86) PCT No.: PCT/KR2024/002522

§ 371 Date: Sep. 4, 2025

(30) Foreign Application Priority Data
Mar. 30, 2023 (KR) 10-2023-0042188

(51) Int. Cl. A61K 39/00; A61K 39/39; A61K 39/395; A61P 35/00; A61P 37/04

(52) U.S. Cl. CPC A61K 39/001106; A61K 39/39; A61K 39/3955; A61P 35/00; A61P 37/04; A61K 2039/55522

(57) ABSTRACT

The present disclosure relates to a combination therapy of a HER2 cancer vaccine and a PD-1 inhibitor or a PD-L1 inhibitor. The combination therapy according to the present disclosure overcomes treatment limitations of the PD-1 inhibitor or the PD-L1 inhibitor and exhibits excellent immunological antitumor activity.

### TECHNICAL FIELD

The present disclosure relates to a novel combination therapy including a HER2 vaccine and an immune checkpoint inhibitor for cancer treatment.

### BACKGROUND ART

A HER2-ICD DNA vaccine is a plasmid-based vaccine containing DNA encoding an intracellular domain of HER2, and the safety and long-term immunological memory efficacy of T-cell immunity have been demonstrated by a Phase 1 clinical study (NCT00436254, JAMA Oncol. 2023; 9(1):71-78). Overexpression of HER2 protein in gastric cancer (GC) is believed to increase the proliferative activity of cancer cells and inhibit apoptosis of cancer cells, and is known to be found in 6% to 30% of gastric cancers. The present inventors have reported in a previous study that the HER2-ICD DNA vaccine has an anti-tumor effect in human gastric cancer cell lines.

The FDA has approved the use of pembrolizumab, which is an antibody treatment for PD-1, in patients previously treated for recurrent locally advanced or metastatic gastric or gastroesophogeal junction cancer patients expressing programmed death-ligand 1 (CPS greater than or equal to 1). Programmed death-1 (PD-1) plays a pivotal role in regulating the activity of T cells, activating the apoptosis of antigen-specific T cells, and inhibiting the apoptosis of regulatory T cells, thereby inhibiting immune responses and promoting self-tolerance. Immunotherapy using immune checkpoint inhibitors such as pembrolizumab, nivolumab, atezolizumab, durvalumab, and avelumab, which prevent this mechanism, has given some patients more survival benefits, but the therapeutic effects seen in clinical trials have limited success rates due to side effects such as lack of existing CD8⁺T cells penetrating tumors, inactivation of immune cells within tumors, and increased activity of immune cells inhibiting immunity.

In addition, acquired immune-related resistance is one of the causes of side effects of immune checkpoint inhibitors. Recently, it has been reported that acquired immune-related resistance occurring in patients who have actually received immune checkpoint inhibitors is associated with an increase in myeloid-derived suppressor cells (MDSC). As the utility of immune checkpoint inhibitors increases, various efforts are required to resolve the problems of immune checkpoint inhibitors described above.

Under the technical background, the present inventors have confirmed that the HER2-ICD DNA vaccine is used in combination with an immune checkpoint inhibitor, particularly a PD-1 inhibitor or a PD-L1 inhibitor, to enhance cancer treatment efficacy while minimizing the problems of non-responsiveness or acquisition resistance to the immune checkpoint inhibitors, thereby completing the present disclosure.

### RELATED ART DOCUMENTS

KR 10-2023-0017640 (HER2 vaccine composition, Publication Date: Feb. 6, 2023)
Mary L Nora Disis et al., JAMA Oncol. 2023 Jan. 1; 9(1):71-78.
Steven H. Sun et al., frontiers in immunology, October 2021, vol. 12, Article 740890

### DISCLOSURE

### Technical Problem

An object of the present disclosure is to provide a pharmaceutical composition for preventing or treating cancer, comprising a vaccine that includes a plasmid containing a polynucleotide sequence encoding a HER2-intracellular domain (hereinafter referred to as a “HER2-ICD DNA vaccine”), and a PD-1 inhibitor or a PD-L1 inhibitor.

In addition, another object of the present disclosure is to provide a combination comprising a HER2-ICD DNA vaccine and a PD-1 inhibitor or a PD-L1 inhibitor.

Further, still another object of the present disclosure is to provide a method for preventing or treating cancer, comprising administering to a patient in need of prevention or treatment of cancer an effective amount of a HER2-ICD DNA vaccine and an effective amount of a PD-1 inhibitor or a PD-L1 inhibitor.

In addition, still another object of the present disclosure is to provide a method for inhibiting progression and/or recurrence of cancer, comprising administering an effective amount of a HER2-ICD DNA vaccine and an effective amount of a PD-1 inhibitor or a PD-L1 inhibitor to a patient in need thereof in order to inhibit progression and/or recurrence of cancer.

In addition, still another object of the present disclosure is to provide a combination of a HER2-ICD DNA vaccine and a PD-1 inhibitor or a PD-L1 inhibitor for simultaneous, separate, or sequential use for prevention or treatment of cancer.

Further, still another object of the present disclosure is to provide a use of a HER2-ICD DNA vaccine and a PD-1 inhibitor or a PD-L1 inhibitor in preparation of a medicament for prevention or treatment of cancer.

In addition, still another object of the present disclosure is to provide a use of a HER2-ICD DNA vaccine and a PD-1 inhibitor or a PD-L1 inhibitor in preparation of a medicament for prevention or treatment of cancer.

Further, still another object of the present disclosure is to provide a kit for preventing or treating cancer, comprising a HER2-ICD DNA vaccine and a PD-1 inhibitor or a PD-L1 inhibitor.

### Technical Solution

The present disclosure will be described in more detail as follows. Meanwhile, descriptions and embodiments described in the present disclosure may be applied to other descriptions and embodiments. That is, all combinations of various elements described in the present disclosure fall within the scope of the present disclosure. In addition, it may not be considered that the scope of the present disclosure is limited by the detailed description described below.

The present disclosure provides a pharmaceutical composition for preventing or treating cancer, comprising a vaccine that includes a plasmid containing a polynucleotide sequence encoding a HER2-intracellular domain (ICD) (hereinafter referred to as a “HER2-ICD DNA vaccine”), and a PD-1 inhibitor or a PD-L1 inhibitor.

### HER2-ICD DNA Vaccine

The HER2-ICD DNA vaccine is a vaccine that includes a plasmid containing a polynucleotide sequence encoding a HER2-intracellular domain, which includes the polynucleotide sequence encoding an amino acid sequence having SEQ ID NO: 2 or at least 80% sequence homology thereto. In other words, the HER2-ICD DNA vaccine is a vaccine that includes a plasmid containing a polynucleotide sequence encoding a HER2-intracellular domain having SEQ ID NO: 2 or a sequence having at least 80% sequence homology thereto.

The HER2-ICD of SEQ ID NO: 2 is an intracellular domain (ICD) of the full-length sequence of HER2 of SEQ ID NO: 1, and is an amino acid sequence at positions 676 to 1255 of the full-length sequence.

More specifically, it may be an amino acid sequence at positions of 676 to 1255 of the full-length sequence of HER2 of SEQ ID NO: 1, or an amino acid sequence having at least 95%, 96%, 97%, 98%, 99% sequence homology thereto.

The vaccine according to the present disclosure may include a polynucleotide sequence encoding an ICD region sequence of HER2, and express the same in vivo so as to kill cancer cells by itself, and may exhibit outstanding therapeutic or preventive effects by virtue of a cancer vaccine such as inhibiting cancer recurrence in a patient from which cancer tissue has been removed by means of surgery or chemical treatment.

The SEQ ID NO: 2 described above may refer to a polynucleotide sequence encoding a sequence comprising or consisting of consecutive amino acids of SEQ ID NO: 2. That is, it may further include an amino acid sequence at an N-terminus and/or C-terminus of SEQ ID NO: 2. The number of amino acid sequences added to the N-terminus and/or C-terminus may be, for example, 15, 14, 13, 12, 11, 10, 9, 8, 7, 6, 5, 4, 3, 2, or 1. When such a sequence is added, it may preferably include the amino acid sequence of the N-terminus and/or C-terminus thereof based on the full-length sequence of HER2 of SEQ ID NO: 1 described above.

In addition, the sequence homology means having at least 85, 90, 91, 92, 93, 94, 95, 96, 97, 98, or 99% or higher homology to the sequence of SEQ ID NO: 2.

More specifically, it may be a sequence having functional homology to a peptide having deletion, insertion and/or substitution of at least one amino acid in the sequence of SEQ ID NO: 2. The functional homology may mean indicating the efficacy of a cancer vaccine desired in the present disclosure.

The amino acid sequence may be modified, for example, by addition, deletion, or substitution of 1, 2, 3, 4, or 5 (i.e., up to 5) amino acids, but compared to a peptide having an unmodified sequence, a polypeptide having a modified sequence may exhibit the identical or increased immunogenicity. It should be understood that the term “identical” does not indicate that the peptide of the modified sequence exhibits significantly reduced immunogenicity or properties as a vaccine compared to the peptide of the unmodified sequence.

Amino acid substitution in proteins and peptides that do not change the activity of molecules as a whole are known in the art. The most common substitution is an substitution between amino acid residues of Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, and Asp/Gly.

The polynucleotide sequence encoding the amino acid sequence of SEQ ID NO: 2 or having at least 80% sequence homology thereto described above is included within the scope of the present disclosure, provided that it is a continuation of codon sequences designed to encode the amino acid sequence of SEQ ID NO: 2 or having at least 80% sequence homology thereto. In other words, the polynucleotide sequence encoding the amino acid sequence of SEQ ID NO; 2 or having at least 80% sequence homology thereto, which may be considered to have functional homology, is included within the scope of the present disclosure.

As used herein, the term “nucleotide” refers to a polymer of deoxyribonucleotide or ribonucleotide present in a single-stranded or double-stranded form. The vaccine may include not only a polynucleotide sequence encoding the amino acid sequence of SEQ ID NO: 2 or having at least 80% sequence homology thereto, but also a sequence complementary to the sequence. The complementary sequence includes not only a perfectly complementary sequence but also a substantially complementary sequence, and refers to a sequence capable of hybridizing to the polynucleotide sequence of a polynucleotide sequence encoding the amino acid sequence of SEQ ID NO: 2 or having at least 80% sequence homology thereto. For example, a protein, which includes a polynucleotide sequence having at least 80%, 90%, 95%, 96%, 97%, 98%, 99% or 100% identity to the polynucleotide encoding the amino acid sequence of SEQ ID NO: 2 or having at least 80% sequence homology thereto, and is encoded by the polynucleotide sequence, substantially retains the functional activity of the protein represented by SEQ ID NO: 2.

A nucleotide sequence encoding the amino acid sequence of SEQ ID NO: 2 or having at least 80% sequence homology thereto may be substituted with a codon having a high expression frequency in a host cell. As used herein, the expression “substituted with a codon having a high expression frequency in a host cell” or “optimized codon” refers to a codon having a high preference according to a host between codons that command an amino acid when DNA is transcribed and translated into a protein in a host cell, and the substitution with the codon having the high preference increases the expression efficiency of an amino acid or a protein encoded by a nucleic acid.

For example, a polynucleotide sequence encoding the amino acid sequence of SEQ ID NO: 2 may be a nucleic acid sequence of SEQ ID NO: 3. In addition, a polynucleotide sequence having an optimized codon, which encodes the amino acid sequence having at least 80%, 85%, 90%, 91%, 92%, 93%, 94%, 95%, 96%, 97%, 98%, or 99% or greater homology to the sequence of SEQ ID NO: 2 is also interpreted to be included within the equivalent range of SEQ ID NO: 3.

In other words, the nucleic acid sequence encoding the HER2-intracellular domain may have 100% homology to the nucleic acid sequence of SEQ ID NO: 3, or may have 99% or higher, 95% or higher, 90% or higher, 85% or higher, or 80% or higher homology thereto. The SEQ ID NO: 3 or a sequence having at least 80% homology thereto is included within the scope of the present disclosure. However, when the homology is limited to the above-described specific values, the immunogenicity against the HER2-ICD due to vaccination has an identical or equivalent effect to that of a sequence having 100% homology.

The plasmid comprises a gene construct in which nucleotides are operably linked to regulatory sequences, thereby enabling the expression of the amino acid sequence of SEQ ID NO: 2 or having at least 80% sequence homology thereto in a host cell. The expression “operably linked to” means that a polynucleotide sequence of interest (e.g., in vivo after administration to a patient) is linked to the regulatory sequence in such a way that expression thereof may be achieved.

In addition, the plasmid may be substituted with a common viral vector. For example, the common viral vector may be a viral vector including a polynucleotide sequence encoding a HER2-intracellular domain having SEQ ID NO: 2 or a sequence having at least 80% sequence homology thereto.

The term “regulatory sequence” has a meaning including a promoter, an enhancer, and/or other regulatory elements (e.g., a polyadenylation signal). The regulatory sequence includes a sequence that directs the expression of a nucleic acid of interest in vivo after administration to a patient, and a sequence that directs the expression of the nucleic acid of interest by a specific signal (e.g., an inducible regulatory sequence).

When the plasmid is administered in vivo, it is possible to express the amino acid sequence of SEQ ID NO: 2 or having at least 80% sequence homology thereto.

For example, possible regulatory sequences that enable expression in mammalian cells include a CMV promoter, SV40, a RSV-promoter (Rous sarcoma virus), a human kidney urea 1α-promoter, a glucocorticoid-inducible MMTV promoter (Moloney mouse tumor virus), and a metallothionein-inducible or tetracycline-inducible promoter.

In addition to the factors that may initiate transcription, the regulatory sequences may include a transcription termination signal such as a β-globin poly-A site, a SV40-poly-A site, or a TK-poly-A site downstream of a polynucleotide according to an embodiment of the present disclosure. In the present disclosure, suitable plasmids are known in the art, and examples thereof include Okayama-Berg cDNA expression plasmids pcDV1 (Parmacia), pRc/CMV, pcDNA1, pcDNA3 (In-vitrogene), pSPORT1 (GIBCO BRL), pGX27, pX, and pUMVC3 (the conventional name is “pNGVL3”, so that both names may be used interchangeably).

The HER2-ICD DNA vaccine of the present disclosure is prepared using pUMVC3, which is a plasmid for preparing a DNA vaccine.

The vaccine that includes the plasmid containing the polynucleotide sequence encoding the HER2-intracellular domain may also be provided in the form of a composition comprising a pharmaceutically acceptable carrier.

The expression “pharmaceutically acceptable” means a substance acceptable to a patient from a pharmacological/toxicological perspective on composition, formulation, safety, etc., and the expression “pharmaceutically acceptable carrier” refers to a medium that does not interfere with the effect of the biological activity of the active ingredient(s), and is non-toxic to a subject at the time of administration.

Typically, proteins, saccharides, polylactic acids, polyglycolic acids, polymeric amino acids, amino acid copolymers, sucrose, trehalose, and the like belong to the pharmaceutically acceptable carriers. Such carriers are well known in the art to which the present disclosure pertains.

The composition may also contain a diluent, such as water, saline, glycerol, or the like. In addition, the composition may be present with auxiliary substances such as wetting or emulsifying agents, pH buffer substances, and the like. A phosphate-buffered saline (PBS) and tromethamine (Tris) without aseptic pyrogens are representative carriers.

The composition may be lyophilized or in aqueous form, for example, an aqueous solution or a suspension. Such a type of liquid formulations is ideal for injection as the composition may be administered directly in a packaged form without the need for reconstitution in an aqueous medium. The composition may be provided in vials or a pre-filled syringe. The syringe may be supplied with or without a needle. The syringe includes a single dose, whereas the vial may include a single dose or multiple doses. In addition, the composition of the present disclosure may be administered using a microneedle.

In addition, the composition may preferably comprise an adjuvant. The adjuvant is any substance that the mixing thereof in the composition increases or modifies the immune response induced by the composition. The adjuvant widely defined is a substance that promotes the immune response.

The suitable adjuvant includes an aluminum salt, such as aluminum hydroxide or aluminum phosphate, may be a salt of calcium, iron or zinc, may be an insoluble suspension of acylated tyrosine or acylated sugar, or may be selected from cationic or anionic derived sugar, and the like, but the present disclosure is not limited thereto.

Examples thereof may include aluminum hydroxide, aluminum phosphate, alum (potassium aluminum sulfate), MF59, virosome, AS04 (mixture of aluminum hydroxide and monophosphoryl lipid A (MPL)), AS03 (mixture of DL-α-tocopherol, squalene and emulsifier polysorbate 80), CpG, Flagellin, Poly I:C, AS01, AS02, ISCOMs, or ISCOMMATRIX.

In addition, the adjuvant may include cytokine used as an immunomodulator. Examples of the cytokine suitable for use as the immunomodulator may be selected from interleukins (e.g., IL-1, IL-2, IL-4, IL-5, IL-6, IL-7, IL-12, etc.), a macrophage colony stimulating factor (M-CSF), a tumor necrosis factor (TNF), a granulocyte macrophage colony stimulating factor (GM-CSF), and the like, but the present disclosure is not limited thereto. In an embodiment of the present disclosure, GM-CSF is used as the adjuvant.

### PD-1 Inhibitor PD-L1 Inhibitor

A PD-1 inhibitor or PD-L1 inhibitor disclosed herein as an example of an immune checkpoint inhibitor includes one that binds to or inhibits PD-1 or PD-L1 with high specificity and affinity, thereby inhibiting the immunosuppressive effect of the relevant signaling pathways.

The PD-1 inhibitor includes nivolumab (Opdivo®), pembrolizumab (Keytruda®), MEDI0680 (AMP-514), CT-011, AMP-224, AMP514 (Amplimmune), BGB-A317, PDR001, REGN2810, JS001, AGEN2034, and variants and biosimilars thereof, but the present disclosure is not limited thereto. Preferably, the PD-1 inhibitor may be nivolumab (Opdivo®) or pembrolizumab (Keytruda®).

Nivolumab is a fully humanized IgG4 (S228P) PD-1 antibody that selectively prevents interaction with PD-1 ligands (PD-L1 and PD-L2), thereby blocking downregulation of anti-tumor T-cell function.

Pembrolizumab is a humanized monoclonal IgG4 kappa antibody directed against PD-1.

The PD-L1 inhibitor includes atezolizumab (Tecentriq® or MPDL3280A), avelumab (Bavencio®), durvalumab (MEDI4736), MPDL3280A (RG7446), BMS-936559 (MDX-1105), MSB0010718C, YW243.55.S70, and variants and biosimilars thereof, but the present disclosure is not limited thereto.

In addition, the PD-1 inhibitor or the PD-L1 inhibitor may be a small molecular material.

The PD-1 inhibitor or the PD-L1 inhibitor may be provided in a composition together with a pharmaceutically acceptable carrier. Typically, proteins, saccharides, polylactic acids, polyglycolic acids, polymeric amino acids, amino acid copolymers, sucrose, trehalose, and the like belong to the pharmaceutically acceptable carriers. Such carriers are well known in the art to which the present disclosure pertains.

In addition, the composition may contain a diluent, such as water, saline, glycerol, or the like. In addition, the composition may be present with auxiliary substances such as wetting or emulsifying agents, pH buffer substances, and the like. A phosphate-buffered saline (PBS) and tromethamine (Tris) without aseptic pyrogens are representative carriers.

Preferably, the PD-1 inhibitor or PD-L1 inhibitor according to the present disclosure may be provided in a liquid formulation or prepared by reconstituting lyophilized powder with sterile water for injection prior to use. Such a type of composition is ideal for injection. The composition may be provided in vials or a pre-filled syringe. The syringe may be supplied with or without a needle. The syringe includes a single dose, whereas the vial may include a single dose or multiple doses. In addition, the composition of the present disclosure may be administered using a microneedle.

### Combination Therapy

A vaccine that includes a plasmid containing a polynucleotide sequence encoding a HER2-intracellular domain according to the present disclosure, and a PD-1 inhibitor or a PD-L1 inhibitor are administered to a subject in need thereof as a combination therapy.

Accordingly, the present disclosure provides a method for preventing or treating cancer, comprising a step of administering to a subject in need thereof a vaccine that includes a plasmid containing a polynucleotide sequence encoding a HER2-intracellular domain having SEQ ID NO: 2 or a sequence having at least 80% sequence homology thereto, and a PD-1 inhibitor or a PD-L1 inhibitor.

These combination therapies are equally applicable to the above-described methods, compositions, combinations, or uses of any combination, unless they are inconsistent with each other.

In the present disclosure, the term “treatment” means not only the result or sign of the result of preventing or reducing tumor growth when a subject is examined by radiation or physical method after performing the combination therapy of the present disclosure or administering the combination or composition of the present disclosure to the subject, but also the result or sign of the result of preventing cancer recurrence/relapse when the subject is examined by radiation or physical method after performing the combination therapy of the present disclosure or administering the combination or composition of the present disclosure to a patient after removing cancer tissues by administering a surgical or anticancer agent to the subject.

In the present disclosure, the term “prevention” means slowing the onset of disease, enhancing the efficacy of an immune checkpoint inhibitor, or reducing the likelihood of developing disease by performing the combination therapy of the present disclosure or administering the combination or composition of the present disclosure to a subject.

In the present disclosure, the term “combination” or “combination therapy” refers to any form of concomitant or parallel treatment with at least two distinct therapeutic agents.

The combination may be two or more separate materials, for example, two separate compositions or two separate collections, a mixture thereof, for example, a single mixture of two or more materials, or variations thereof.

In general, elements of the combination are functionally associated or related. This may be administered at the same time, or separately within a time interval that allows the therapeutic agents to exert a cooperative effect, and may optionally be used in a combination of non-fixed doses, packaged together with instructions on the combined administration.

For example, the combination according to the present disclosure may be administered separately or sequentially within a time interval that enables the combination to exhibit synergistic and/or complementary effects. That is, the vaccine that includes a plasmid containing a polynucleotide sequence encoding a HER2-intracellular domain, and the PD-1 inhibitor or the PD-L1 inhibitor may be administered to a patient in separate dosage forms, and a route of the administration may be the same or different.

In the present disclosure, “simultaneous” administration means separately administering each of the HER2-ICD DNA vaccine and the PD-1 inhibitor or PD-L1 inhibitor to a patient at the same time or at different times. For example, “simultaneous” administration means that each of the HER2-ICD DNA vaccine and the PD-1 inhibitor or the PD-L1 inhibitor is administered independently at substantially the same time, or separately within a time interval such that the HER2-ICD DNA vaccine and the PD-1 inhibitor or the PD-L1 inhibitor may exhibit a cooperative therapeutic effect.

In the present disclosure, “separate” administration means administering each of the HER2-ICD DNA vaccine and the PD-1 inhibitor or the PD-L1 inhibitor to a patient simultaneously, substantially concurrently, or sequentially in any order from non-fixed dose dosage forms. There may or may not be a specified time interval for administration of each of the HER2-ICD DNA vaccine and the PD-1 inhibitor or the PD-L1 inhibitor.

In the present disclosure, “sequential” administration means administering each of the HER2-ICD DNA vaccine and the PD-1 inhibitor or the PD-L1 inhibitor to a patient in a separate operation from non-fixed (separate) dose dosage forms.

In the present disclosure, the term “patient” or “subject” refers to a living organism suffering from, having been treated for, or susceptible to a condition, such as cancer, which may be prevented or treated by the embodiments of the composition, combination, use, or method of the present disclosure, and includes both humans and animals. The subject includes mammals (e.g., mice, monkeys, horses, cows, pigs, dogs, cats, etc.), but is not limited thereto, and is preferably a human. In addition, the term “patient” or “subject” in the present disclosure includes a cancer patient or any subject capable of having cancer inherently without distinction.

In the present disclosure, the term “administration” refers to the introduction of a composition, combination, or the like to a subject using any of various methods and delivery systems known in the art to which the present disclosure pertains. Exemplary routes of administration include intravenous, intramuscular, subcutaneous, intradermal, intraperitoneal, spinal, or other parenteral routes of administration, such as by injection or infusion. Other parenteral routes include local, epithelial, or mucosal routes of administration, such as intranasal, vaginal, rectal, or sublingual routes. In addition, each drug may be administered via a different route.

The inventors of the present disclosure have conducted an experiment of a recurrence rate or a survival rate when the HER2-ICD vaccine and pembrolizumab are co-administered to a patient with gastric cancer or breast cancer (a patient with HER2 expression) by adjuvant therapy after surgery, and expect an increased recurrence rate or survival rate through co-administration of the HER2-ICD vaccine and pembrolizumab.

Thus, the combination therapy of the present disclosure not only means an effect of preventing or reducing tumor growth when administered to a subject or patient, but also prevents cancer recurrence or growth, minimizes side effects of immune checkpoint inhibitors, and minimizes the occurrence of acquired immune-related resistance due to the use of immune checkpoint inhibitors.

The HER2-ICD DNA vaccine according to the present disclosure allows a subject or patient to initiate an immune response against a HER2-ICD antigen such that the immune system of the subject may target and destroy cells expressing the same antigen, thereby reducing cancer progression or cancer recurrence in the subject. In addition, the HER2-ICD DNA vaccine minimizes the generation of acquired immune-related resistance to maximize the treatment efficacy of the immune checkpoint inhibitor, minimize the dosage thereof, and minimize resistance to the immune checkpoint inhibitor.

Specifically, the combination therapy according to the present disclosure has confirmed that the antigen-specific immune response is actually activated by increasing a CD4⁺IFN-γ⁺/CD8⁺IFN-γ⁺ ratio through the combination of the HER2-ICD DNA vaccine, and also confirmed that the combination therapy inhibited activated immune cells and inhibited the increase in myeloid-derived suppressor cells (MDSCs) known to play an important role in immune evasion of cancer cells.

Among the immune checkpoint inhibitors, in particular, the PD-1 inhibitor or the PD-L1 inhibitor has MDSCs that are generally increased to maintain immunological homeostasis as the inflammatory process of a tumor site is activated. This increase in MDSCs may lead to an imbalance of immune cells in the tumor microenvironment, thereby contributing to the development of acquired immune-related resistance. In addition, it has been reported that MDSCs increase even in peripheral blood of a group of melanoma patients treated with an actual immune checkpoint inhibitor. The combination therapy according to the present disclosure may inhibit the increase in MDSCs and the like, thereby inhibiting the generation of acquired immune-related resistance and maximizing the effects of immune checkpoint inhibitors even at low doses. That is, it may reduce the fraction of MDSC cells, which are immunosuppressive cells.

In addition, the combination therapy according to the present disclosure increases the fraction of T cells in tumor tissues, particularly cytotoxic T cells, specifically CD8⁺T cells and CD8⁺IFN-γ⁺T cells, through the combination with HER2-ICD DNA vaccines. In the case of the immune checkpoint inhibitor, it is preferable to exhibit a direct anti-tumor effect in tumor tissues by controlling immune activity in tumor tissues. In this regard, the combination with the HER2-ICD DNA vaccine significantly increases the distribution or activity of cytotoxic T cells in tumor tissue, thereby maximizing the action efficacy of the immune checkpoint inhibitor even at low doses. That is, it may increase or activate an amount of cytotoxic T cells in tumor tissue.

In other words, the combination therapy according to the present disclosure may reduce the therapeutic dose of a drug for exhibiting therapeutic efficacy and may enhance the effect of treating/preventing cancer.

For the method, composition, combination, or the use of any combination described above according to the present disclosure, the HER2-ICD DNA vaccine and the PD-1 inhibitor or PD-L1 inhibitor described above are administered in a therapeutically effective amount.

The therapeutically effective amount in the present disclosure refers to a dosage of a composition or combination comprising a HER2-ICD DNA vaccine and a PD-1 inhibitor or PD-L1 inhibitor required to inhibit tumor cell growth, eliminate cancer, or slow down or stop the progression thereof in a patient.

The HER2-ICD DNA vaccine according to the present disclosure may be administered to a subject on any schedule suitable to induce and/or maintain a therapeutic effect on cancer growth or recurrence. In addition, the HER2-ICD DNA vaccine may be administered simultaneously, sequentially, or alternately with the administration of the PD-1 inhibitor or the PD-L1 inhibitor. That is, the HER2-ICD DNA vaccine may be administered on any schedule to improve or boost the effects of the PD-1 inhibitor or the PD-L1 inhibitor under clinical determination.

The frequency of administration may be several days, weeks, or months. For example, the frequency may be one or more times a week, for example, twice per week, three times per week, four times per week, five times per week, six times per week, or every day. The frequency may also be 1, 2, 3, or 4 weeks. The specific frequency is a function of the particular disease or condition being treated.

According to some embodiments of the present disclosure, the HER2-ICD DNA vaccine may be administered to a patient three or more times per month, with additional booster shots as needed.

Preferably, the HER2-ICD DNA vaccine may be administered intradermally or together with an adjuvant.

The PD-1 inhibitor or PD-L1 inhibitor according to the present disclosure may be administered to a subject on any schedule suitable to induce and/or maintain a therapeutic effect on cancer growth or recurrence. The exact dosage and duration of treatment required to treat a patient with the PD-1 inhibitor or PD-L1 inhibitor may be determined by a physician taking into account the stage and severity of the disease as well as the specific needs and responses of each individual patient.

Preferably, the PD-1 inhibitor or the PD-L1 inhibitor may be administered by intravenous, intramuscular, intradermal, or subcutaneous administration.

In the combination therapy, each therapeutic agent may be administered concurrently (i.e., with the same agent), concurrently (i.e., with a separate agent that is administered one after the other in any order), or sequentially in any order. Sequential administration may mean that the therapeutic agents in the combination therapy are in different dosage forms, are administered on different dosing schedules, or are administered to different parts of the body.

Such combination therapy may allow the subject to have improved advantages over monotherapy, for example, lower doses, less frequent doses, and/or shorter treatment periods.

The disease requiring treatment of the combination therapy of the present disclosure is cancer. More specifically, the cancer may be selected from the group consisting of breast cancer, brain cancer, nerve cancer, colorectal cancer, lung cancer, non-small cell lung cancer, small cell lung cancer, stomach cancer, liver cancer, blood cancer, bone cancer, pancreatic cancer, skin cancer, head or neck cancer, cutaneous or intraocular melanoma, uterine cancer, ovarian cancer, rectal cancer, perianal cancer, colon cancer, breast cancer, fallopian tube carcinoma, endometrial carcinoma, cervical cancer, vaginal cancer, vulvar carcinoma, Hodgkin's disease, esophageal cancer, small intestinal cancer, endocrine cancer, thyroid cancer, parathyroid cancer, adrenal cancer, soft tissue sarcoma, urethral cancer, penile cancer, prostate cancer, chronic or acute leukemia, lymphocytic lymphoma, bladder cancer, kidney or ureter cancer, renal cell carcinoma, renal pelvic carcinoma, central nervous system (CNS) tumor, primary CNS lymphoma, spinal cord tumor, brainstem glioma, and pituitary adenoma.

More specifically, the disease may be breast cancer or gastric cancer.

More specifically, the disease may be HER2-positive cancer. The term “HER2-positive” means a cancer cell that expresses a HER2 protein or gene, and the degree of expression is irrelevant.

The present disclosure provides a combination comprising a HER2-ICD DNA vaccine and a PD-1 inhibitor or a PD-L1 inhibitor.

The present disclosure provides a method for preventing or treating cancer, comprising administering to a patient in need of prevention or treatment of cancer an effective amount of a HER2-ICD DNA vaccine and an effective amount of a PD-1 inhibitor or a PD-L1 inhibitor.

The present disclosure provides a method for inhibiting progression and/or recurrence of cancer, comprising administering an effective amount of a HER2-ICD DNA vaccine and an effective amount of a PD-1 inhibitor or a PD-L1 inhibitor to a patient in need thereof in order to inhibit progression and/or recurrence of cancer.

The present disclosure provides a pharmaceutical composition comprising a HER2-ICD DNA vaccine and a PD-1 inhibitor or a PD-L1 inhibitor for use in preventing or treating cancer.

The present disclosure provides a combination of a HER2-ICD DNA vaccine and a PD-1 inhibitor or a PD-L1 inhibitor for simultaneous, separate, or sequential use for prevention or treatment of cancer.

The present disclosure provides a use of a HER2-ICD DNA vaccine and a PD-1 inhibitor or a PD-L1 inhibitor in preparation of a medicament for prevention or treatment of cancer.

The present disclosure provides a use of a HER2-ICD DNA vaccine and a PD-1 inhibitor or a PD-L1 inhibitor in preparation of a medicament for prevention or treatment of cancer.

The present disclosure provides a kit for preventing or treating cancer, comprising a HER2-ICD DNA vaccine and a PD-1 inhibitor or a PD-L1 inhibitor.

The HER2-ICD DNA vaccine and the PD-1 inhibitor or the PD-L1 inhibitor may be formulated to suit the route of administration and provided as a kit as needed.

### Advantageous Effects

The combination therapy of the HER2 cancer vaccine and the PD-1 inhibitor or the PD-L1 inhibitor according to the present disclosure inhibits generation of acquired immune-related resistance and maximizes functional efficacy of the immune checkpoint inhibitor even at low doses. In particular, the combination therapy of the HER2 cancer vaccine and the PD-1 inhibitor or the PD-L1 inhibitor according to the present disclosure exhibits excellent efficacy in treating cancer diseases by inhibiting an increase in MDSC according to administration of the immune checkpoint inhibitor, increasing an interferon gamma (IFN-γ) positive CD4+T cell fraction, and greatly enhancing the fraction and activity of cytotoxic T cells in tumor tissues.

### DESCRIPTION OF DRAWINGS

FIG. 1 shows a drug administration schedule after a tumor is formed in a gastric cancer xenograft mouse model transplanted with HuCD34⁺.

FIG. 2 shows changes in relative tumor volume (RTV) and tumor growth inhibition (TGI) of groups (G1 to G3) according to days elapsed after drug administration.

FIG. 3 shows results of confirming an activation ratio of CD4⁺T cells and CD8⁺T cells in the spleen of mice belonging to each group by performing flow cytometry, as a representative flow cytometry plot. (Pem=Pembrolizumab, mpk=milligram per kilogram (mg/kg))

FIG. 4 shows results of confirming a ratio of myeloid-derived suppressor cells (MDSCs) in the spleen of mice belonging to each group by performing flow cytometry, as representative flow cytometry plot.

FIG. 5 shows results of confirming a ratio of CD4⁺T cells and CD8⁺T cells distributed in tumor tissues isolated from each group of mice by performing flow cytometry, as representative flow cytometry plot.

FIG. 6 shows results of confirming a ratio of CD4⁺IFN-γ⁺T cells and CD8⁺IFN-γ⁺T cells distributed in tumor tissues isolated from each group of mice by performing flow cytometry, as representative flow cytometry plot.

### BEST MODE

Hereinafter, the present disclosure will be described in more detail through examples. These examples are only for more specifically describing the present disclosure, and it will be apparent to those skilled in the art that the scope of the present disclosure is not limited by these examples according to the gist of the present disclosure.

### Experimental Example 1. NCI-N87 Cell Line Culture

NCI-N87 cell lines were cultured in a constant temperature and humidity incubator at 37° C. in the presence of 5% CO₂ using a cell culture medium (RPMI-1640, GIBCO) containing 10% fetal bovine serum (GIBCO) and 1% penicillin/streptomycin (GIBCO) inactivated at 56° C. for 30 minutes. The cell lines were subcultured by treating 0.25% Trypsin-EDTA (GIBCO) and maintained at 50% confluency.

In addition, a mycoplasma detection test was performed in a medium cultured for 3 days before tumor formation, and a cell line in which mycoplasma was not detected was cultured and used in the experiment.

### Experimental Example 2. Preparation of Gastric Cancer Xenograft Mouse Model Transplanted with HuCD34⁺

As a mouse model, humanized NGS (NOD scid gamma mouse, The Jackson Laboratory) mice transplanted with HuCD34⁺ hematopoietic stem cell (HSC) were used. The animal experiment complied with guidelines for animal experimental ethics of the Gwangju Institute of Science and Technology and was conducted in accordance with the Animal Experiment Plan (GIST-2022-034).

The NCI-N87 cell lines cultured in Experimental Example 1 were treated with 0.25% Trypsin-EDTA to separate the cells, and suspended in a buffer mixed with 50 μL of PBS and 50 μL of Matrigel (BD Biosciences). Tumors were formed in the left flank of the shaved mouse by subcutaneous implantation at an amount of 1×10⁷ cell per CD34⁺ NSG mouse. The tumor size of each mouse was measured, and when the size reached 100 to 200 mm³, the mice were randomly divided into experimental groups. The administered drugs, dosages, administration methods, and number of mice in each group are shown in Table 1 below.

TABLE 1

| | | Dose | | | |
|---|---|---|---|---|---|
| | | Level | Dose | Dose | No. of |
| Group | Treatment | (mg/kg) | Route | Frequency | Animals |
| G1 | Vehicle | 0 | ID | QW/4 | 4 |
| | | | | Times | |
| G2 | [HER2 - ICD DNA | 105 μg/ | ID⁺IV | QW/4 | 4 |
| | Vaccine⁺GM-CSF] + | head + | | Times | |
| | Pembrolizumab | 5 mg/kg | | QW/3 | |
| | | | | Times | |
| G3 | Pembrolizumab | 5 mg/kg | IV | QW/3 | 4 |
| | | | | Times | |

*Vehicle: 0.9% Normal saline, QW: Once a week

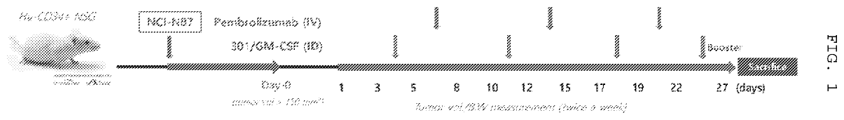

As shown in Table 1, the HER2-ICD DNA vaccine (100 μg) was mixed with rhuGM-CSF (5 μg) and administered intradermally a total of 4 times (once a week) (G2). In addition, a negative control group (G1) to which only vehicle was assigned to evaluate the efficacy, and for comparison with a co-administration group the HER2-ICD DNA vaccine and pembrolizumab (5 mg/kg), low-dose (5 mg/kg) and high-dose (10 mg/kg) of single-dose groups (G3 and G4, respectively, as positive control groups) were additionally assigned. The HER2-ICD DNA vaccine and rhuGM-CSF were administered intradermally by removing the fur of the right flank of the mouse, disinfecting the mouse with 70% alcohol, and intradermal injection was performed using an insulin syringe (31 G, 1 ml), and pembrolizumab was administered by tail vein injection using the insulin syringe (31 G, 1 ml) a total of three times (once a week). The drug administration schedule after tumor formation is shown in FIG. 1.

### Experimental Example 3. Measurement of Weight and Tumor Size of Mouse Model

The body weight and tumor size of the mice belonging to each group were measured at an interval of 3 days (at least 2 times/week). In this case, the tumor size was followed the equation of “Tumor size (mm³)=(length×width²)/2”. The relative tumor volume (RTV) was shown by measuring the tumor size at the time of drug administration and the tumor size at the end of the experiment, and calculating a ratio thereof (RTV=(tumor volume on measured day)/(tumor volume on day 0)).

### Experimental Example 4. Sample Separation After End of Experiment

After 25 days from the start of drug administration, all the experiments were completed, and the tumor was removed from the mice belonging to each group and subject to a single cell process using tumor dissociation kit (Miltenyi Biotec), and tumor infiltrated lymphocytes were isolated and preserved in a cell preservative (Cell banker, amsbio). Simultaneously, splenocytes from which red blood cells were removed and leukocytes were isolated from the spleen of the mice belonging to each group and lyophilized. In addition, after thawing, a ratio of immune cells for each phenotype was investigated by performing flow cytometry analysis with a cell surface marker label on living cells.

### Experimental Example 5. Flow Cytometry Analysis

Human antibodies anti-CD45 (clone RPA-T4), anti-CD3 (clone HIT), BV421 binding anti-CD4 (clone RPA-T4), anti-CD8 (clone RPA-T4), protein transport inhibitor (GolgiPlug), and staining buffer used for flow cytometry were purchased from BD Bioscience. eFlor780 Binding Fixable Viability dye was purchased from ThermoFisher.

For flow cytometry, 2,000,000 cells per well were cultured in 96× round-bottom well-plates. Specifically, the cells were cultured for 72 hours in a 37° C. incubator in the presence of 1 μg/ml of anti-CD3. As the culture medium, a complete RPMI1640 medium containing 10% fetal bovine serum, 10 mM HEPES, 50 PM β-ME, and penicillin/streptomycin/L-glutamine was used.

To sort viable cells only, the cells were labeled with eFlor780 binding Fixable Viability dye in PBS at a concentration of 1/100 at 4° C. for 15 minutes and washed twice with a staining buffer after 15 minutes. After washing, anti-CD45, anti-CD3⁺, anti-CD4⁺, and anti-CD8⁺ antibodies were labeled on the cell surface at 4° C. for 30 minutes in the staining buffer at a concentration of 1/100. The cells labeled with cell surface proteins were washed twice with the staining buffer. Thereafter, the labeled cells were washed twice with the staining buffer, and flow cytometry was performed.

Flow cytometry analysis was performed using Attune NxT Acoustic Focusing Cytometer (Invitrogen), and data analysis was performed using Flowjo v10.7.1 software (FlowJo). Statistical analysis was performed using a GraphPad Prism (GraphPad Software, San Diego, Calif., USA). The significance between groups was verified through the Dunnett's multiple comparison method (One-way ANOVA).

### Example 1. Changes in Weight and Tumor Size of Mouse Model

The changes in the body weight of the mice belonging to the groups G1 to G3 from the start of the experiment to the end of the experiment are shown in Table 2 below.

TABLE 2

| | Initial Body | Body Weights at | Body Weight |
|---|---|---|---|
| Group | Weights (g) | sacrifice (g) | Change (%) |
| G1 | 22.45 ± 1.02 | 20.43 ± 0.81 | −10.07 |
| G2 | 22.93 ± 0.32 | 20.00 ± 0.61 | −12.61 |
| G3 | 23.00 ± 0.92 | 19.03 ± 0.18 | −19.17 |

Meanwhile, the changes in the tumor size of the mice belonging to the groups G1 to G3 between the experiment end date and the experiment start date are shown in Table 3 below.

TABLE 3

| | Absolute Tumor | Relative Tumor | |
|---|---|---|---|
| | volume (mm³) at | volume (mm³) at | TGI rate (%) |
| Group | sacrifice | sacrifice | at Day 25 |
| G1 | 1075.68 ± 152.71 | 1223.99 ± 607.57 | — |
| G2 | 685.46 ± 238.56 | 626.86 ± 201.20 | 49 |
| G3 | 1013.72 ± 41.01 | 788.09 ± 179.84 | 36 |

$${}^{*} TGI ( Tumor growth inhibition rate ) = [ 1 - ( RTV of the treated group ) / ( RTV of the control group ) ] \times 100 ( \% )$$

G2 and G3 showed a tumor growth inhibition rate (TGI) of 49% or 36%, respectively, compared to the negative control group G1 (day 25).

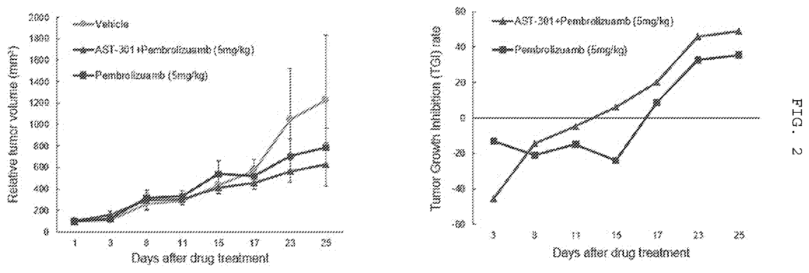

Specifically, as a result of observing the changes in RTV and TGI of each group according to the elapsed days after drug administration (FIG. 2), the tumor cell growth inhibitory effect was shown in G3, which is a single administration group of pembrolizumab. In addition, as G2 showed a higher TGI value than G3, it was confirmed that the co-administration with the HER2-ICD DNA vaccine was more effective than the single administration of pembrolizumab under the condition of the same dose of pembrolizumab.

In other words, it was confirmed that the tumor growth inhibitory effect was enhanced by co-administering the two immune anticancer drugs (pembrolizumab and HER2-ICD DNA vaccine).

### Example 2. Immune-Profiling Analysis Using Spleen Cells

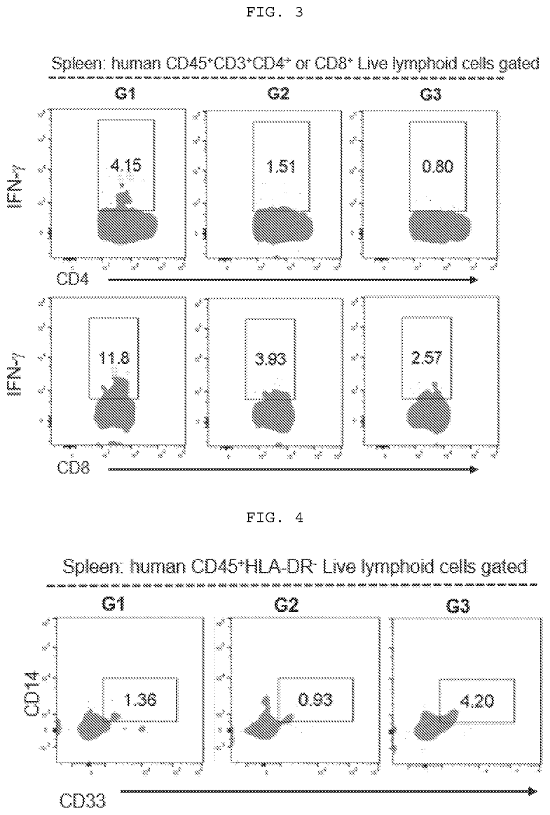

The spleens of mice belonging to each group were isolated according to Experimental Example 4 and flow cytometry was performed to observe a ratio of CD4⁺TIFN-γ⁺ cells and CD8⁺IFN-γ⁺T cells and MDSC. The results are shown in FIGS. 3 and 4 as a representative flow cytometry plot, and the numerical values of each group are summarized and shown in Table 4 below.

TABLE 4

| | | MDSC |
|---|---|---|
| | CD4⁺IFN-γ⁺/ | (CD45⁺HLA-DR⁻ |
| | CD8⁺IFN-γ⁺ | CD33⁺CD14⁺) |
| Group | (relative value) | (relative value) |
| G1 | 100% | 100% |
| G2 | 125% | 49% |
| G3 | 91% | 118% |

As shown in Table 4, CD4⁺IFN-γ⁺/CD8⁺IFN-γ⁺ ratio was decreased in the pembrolizumab single administration group, whereas CD4⁺IFN+/γ-CD8⁺IFN-γ⁺ ratio was increased in the co-administration group of the HER2-ICD DNA vaccine and pembrolizumab (5 mg/kg). In addition, the pembrolizumab single administration group (G3) showed an increase in MDSC fraction, but the co-administration with the HER2-ICD DNA vaccine (G2) showed a rapid decrease as compared to that in G1. The result may predict that the co-administration of two drugs with different mechanisms of action may have a long-term anticancer effect while increasing the CD4⁺IFN-γ⁺T cell fraction, which plays a long-term role, in exhibiting an anticancer effect.

In addition, it is known that MDSC is increased to maintain immunological homeostasis while the inflammatory process of the tumor site is activated due to the administration of pembrolizumab, which is an immune checkpoint inhibitor (ICI), and by confirming that MDSC is decreased in the co-administration group of the HER2-ICD DNA vaccine and pembrolizumab, it was confirmed that the HER2-ICD DNA vaccine may contribute to the immunological change in a direction of decreasing MDSC increased by pembrolizumab.

### Example 4. Immune-Profiling Analysis of Tumor Tissue

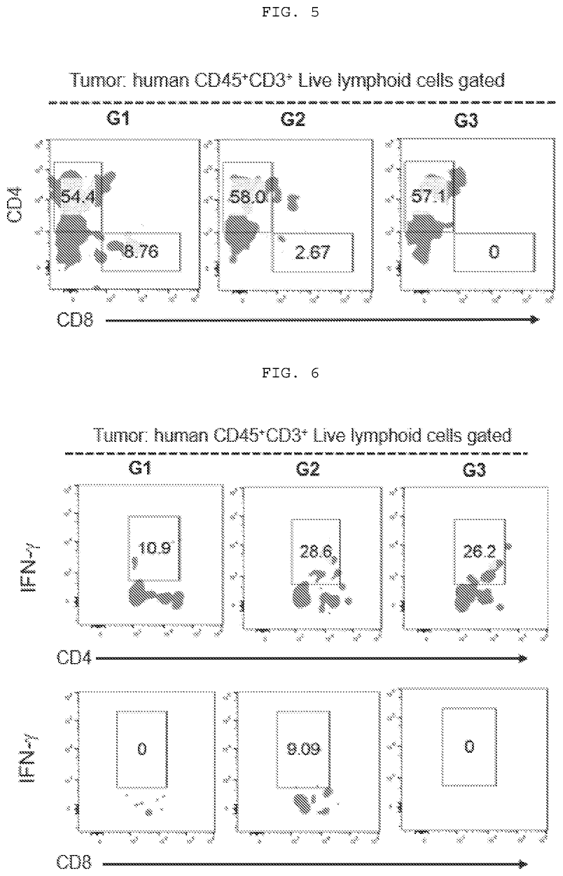

Flow cytometry was performed on a tumor tissue isolated from each group of mice according to Experimental Example 4, and a ratio of CD4⁺T cells, CD4⁺IFN-γ⁺T cells, CD8⁺T cells, and CD8⁺IFN-γ⁺T cells present in the tumor tissue was observed. Among them, results for CD4⁺T cells and CD8⁺T cells are shown in FIG. 5, and results for CD4⁺IFN-γ⁺T cells and CD8⁺IFN-γ⁺T cells are shown in FIG. 6. The numerical values of each group are summarized and shown in Table 4 below. The values are the percentage of cells positive for the marker in the tumor or relative values based on the negative control group.

TABLE 5

| | | CD4⁺ | | CD8⁺ % | |
|---|---|---|---|---|---|
| | | % of | | % of | |
| | CD4⁺ | CD45⁺ | CD8⁺ % | CD45⁺CD3⁺ | |
| | % of | CD3⁺CD4⁺ | % of | CD8⁺ | CD4⁺ % of |
| | CD45⁺ | (relative | CD45⁺CD3⁺ | (relative | CD45⁺CD3⁺CD4⁺IFN- |
| Group | CD3⁺CD4⁺ | value) | CD8⁺ | value) | γ⁺ |
| G1 | 50.35% | 100% | 7.52% | 100% | 26.88% |
| G2 | 49.53% | 98.4% | 14.13% | 188% | 27.5% |
| G3 | 56.37% | 112% | 0.45% | 5.9% | 30.53% |

| | | | CD8⁺ % |
|---|---|---|---|
| | CD4⁺ % of | | % of |
| | CD45⁺CD3⁺CD4⁺IFN- | CD8⁺ % | CD45⁺CD3⁺CD8⁺IFN- |
| | γ⁺ | % of | γ⁺ |
| | (relative | CD45⁺CD3⁺CD8⁺IFN- | (relative |
| Group | value) | γ⁺ | value) |
| G1 | 100% | 16.05% | 100% |
| G2 | 102% | 8.23% | 51.28% |
| G3 | 113.6% | 0% | 0% |

As shown in Table 5, in the pembrolizumab single administration group (G3), CD8⁺T cells and CD8⁺IFN-γ⁺T cells present in tumor tissue were hardly confirmed. However, when co-administrated with the HER2-ICD DNA vaccine (G2), the tumor tissue distribution of the cells was increased.

Therefore, it was confirmed through the experiment that the co-administration of the HER2-ICD DNA vaccine and pembrolizumab (5 mg/kg) may improve the anti-tumor effect by enhancing the intratumoral distribution of cytotoxic T cells that contribute to direct killing of cancer cells.

Through the results, it was confirmed that the combination therapy according to the present disclosure may overcome the therapeutic limitations of the PD-1 inhibitor or the PD-L1 inhibitor and enhance the immunological anticancer activity.

Although the particulars of the present disclosure have been described in detail, it will be obvious to those skilled in the art that such particulars are merely preferred embodiments and are not intended to limit the scope of the present disclosure. Therefore, the substantive scope of the present disclosure is defined by the appended claims and their equivalents.

1-15. (canceled)

16. A method for preventing or treating cancer, comprising administering to a subject in need thereof a vaccine that includes a plasmid containing a polynucleotide sequence encoding a HER2-intracellular domain having SEQ ID NO: 2 or a sequence having at least 80% sequence homology thereto, and a PD-1 inhibitor or a PD-L1 inhibitor.

17. The method of claim 16, wherein the PD-1 inhibitor is any one selected from the group consisting of nivolumab, pembrolizumab, MEDI0680 (AMP-514), CT-011, AMP-224, AMP514, BGB-A317, PDR001, REGN2810, JS001, and AGEN2034.

18. The method of claim 17, wherein the PD-1 inhibitor is pembrolizumab.

19. The method of claim 16, wherein the PD-L1 inhibitor is any one selected from the group consisting of atezolizumab, avelumab, durvalumab, MPDL3280A, BMS-936559, MSB0010718C, and YW243.55.S70.

20. The method of claim 16, wherein the polynucleotide sequence encoding the HER2-intracellular domain having SEQ ID NO: 2 or a sequence having at least 80% sequence homology thereto is SEQ ID NO: 3 or a sequence having at least 80% sequence homology thereto.

21. The method of claim 16, wherein the polynucleotide sequence encoding the HER2-intracellular domain having SEQ ID NO: 2 is SEQ ID NO: 3 or a sequence having at least 80% sequence homology thereto.

22. The method of claim 16, wherein the polynucleotide sequence encoding the HER2-intracellular domain having SEQ ID NO: 2 or a sequence having at least 80% sequence homology thereto is SEQ ID NO: 3.

23. The method of claim 16, wherein the vaccine that includes a plasmid containing a polynucleotide sequence encoding the HER2-intracellular domain having SEQ ID NO: 2 is SEQ ID NO: 3 or a sequence having at least 80% sequence homology thereto, and the PD-1 inhibitor is pembrolizumab.

24. The method of claim 16, wherein the vaccine that includes a plasmid containing a polynucleotide sequence encoding the HER2-intracellular domain having SEQ ID NO: 2 or a sequence having at least 80% sequence homology thereto is SEQ ID NO: 3, and the PD-1 inhibitor is pembrolizumab.

25. The method of claim 16, wherein the vaccine further comprises an adjuvant.

26. The method of claim 25, wherein the adjuvant is GM-CSF.

27. The method of claim 16, wherein the cancer is any one selected from the group consisting of breast cancer, brain cancer, nerve cancer, colorectal cancer, lung cancer, non-small cell lung cancer, small cell lung cancer, stomach cancer, liver cancer, blood cancer, bone cancer, pancreatic cancer, skin cancer, head or neck cancer, cutaneous or intraocular melanoma, uterine cancer, ovarian cancer, rectal cancer, perianal cancer, colon cancer, breast cancer, fallopian tube carcinoma, endometrial carcinoma, cervical cancer, vaginal cancer, vulvar carcinoma, Hodgkin's disease, esophageal cancer, small intestinal cancer, endocrine cancer, thyroid cancer, parathyroid cancer, adrenal cancer, soft tissue sarcoma, urethral cancer, penile cancer, prostate cancer, chronic or acute leukemia, lymphocytic lymphoma, bladder cancer, kidney or ureter cancer, renal cell carcinoma, renal pelvic carcinoma, central nervous system (CNS) tumor, primary CNS lymphoma, spinal cord tumor, brainstem glioma, and pituitary adenoma.

28. The method of claim 27, wherein the cancer is breast cancer or gastric cancer.

29. The method of claim 16, wherein the cancer is HER2-positive cancer.

30. A combination comprising a vaccine that includes a plasmid containing a polynucleotide sequence encoding a HER2-intracellular domain having SEQ ID NO: 2 or a sequence having at least 80% sequence homology thereto, and a PD-1 inhibitor or a PD-L1 inhibitor.
